(19) United States
(12) Patent Application Publication
(10) Pub. No.: US 2005/0016534 A1
(43) Pub. Date: Jan. 27, 2005

(54) User interface for a medical ventilator

(76) Inventor: Mats Ost, Taberg (SE)

Correspondence Address: SCHIFF HARDIN LLP;Patent Department, 6600 Sears Tower, 233 South Wacker Drive, Chicago, IL, 60606, US

(21) Appl. No.: 10/872,171

(22) Filed: Jun. 18, 2004

(30) Foreign Application Priority Data
Jun. 18, 2003 (SE) 0301767-0

(51) Int. Cl. A61M 16/00

(52) U.S. Cl. 128/204.18; 128/204.21

(57) ABSTRACT

A user interface for a medical ventilator has a screen adapted to display curves generated by a control unit representing measured parameters for the medical ventilator, and an input arrangement allowing a user to enter target values for control parameters for the medical ventilator. Simplified modification or programming of the medical ventilator is achieved by adaptation of the screen to display curves and input target values in a volume-pressure graph.

### BACKGROUND OF THE INVENTION

1. Field of the Invention

The present invention is relates to a user interface for a medical ventilator.

2. Description of the Prior Art

The user interface is an important component of a medical ventilator, and normally include a screen which can be used to display numerical and graphical information related to operating parameters, ventilation modes, monitored parameters, respiration curves, etc. One such interface is described in U.S. Pat. No. 5,881,723.

It is also known to provide a medical ventilator with a user interface having an interactive screen. An example of such a ventilator is the Servo® ventilator from Siemens Elema AB, Sweden (now Maquet Critical Care AB). This user interface has an interactive screen which selectively can be used for programming of functions and as a monitor to display breathing curves and other information.

In the present context programming of functions means primarily breathing modes, where the parameter values can be input and numerically displayed on a screen.

It is desirable to have a user interface which enables a simple, intuitive and user friendly handling with respect to both input of target values relevant for the treatment given and understanding of the condition of the patient from displayed measured information. The risk of any errors occurring due to the interaction between user and machine can then be reduced to a minimum.

### SUMMARY OF THE INVENTION

An object of the present invention is to provide a user interface for a medical ventilator of the above type which at least partly addresses the above stated problems and desires.

With a presentation of curves and input target values in a volume-pressure graph several advantages are achieved.

The user is provided with an immediate sense for what the input target values represent in relation to the treatment to be given. The user can get a proper intuitive feeling for the relationship between volume and pressure, as compared to numerical or graphical time-dependent displays.

The volume-pressure curve(s) provides an unambiguous, easily seen variation of the progress of the treatment breath-by-breath. This due to the natural repetitiveness of the curve (the curve of one breath essentially forms a closed oval-shaped Figure), which makes it much easier to spot even minor deviations as compared to time-based representations of volume or pressure, where two consecutive curves must be compared.

### DESCRIPTION OF THE DRAWINGS

FIG. 1 schematically illustrates an exemplary embodiment of a medical ventilator having a user interface according to the invention.

FIG. 2 shows a first example of a volume-pressure graph displayed on an interactive screen in the user interface according to FIG. 1.

FIG. 3 shows a second example of a volume-pressure graph displayed on an interactive screen in the user interface.

FIG. 4 shows a third example of a volume-pressure graph displayed on an interactive screen in the user interface.

FIG. 5 shows a fourth example of a volume-pressure graph displayed on an interactive screen in the user interface.

### DESCRIPTION OF THE PREFERRED EMBODIMENTS

With reference to FIG. 1, an exemplary embodiment of a medical ventilator 2 is shown. The medical ventilator 2 has a pneumatic unit 4 for the preparation of a breathing gas. In this present case the pneumatic unit 4 has two gas inlets 6A, 6B for the coupling in of two gases, for example oxygen and air.

The prepared breathing gas is carried toward a patient 8 via an inspiration line 10 during inspiration and away from the patient 8 via an expiration line 12 during expiration.

The medical ventilator 2 further has a control unit 14 for regulation and control of the pneumatic unit 4 and a user interface 16 according to the invention, through which an operator can enter suitable target values for the treatment of the patient 8.

The user interface 16 in this embodiment has an interactive screen 18. To increase safety against unwanted changes or settings a function switch 20 may be included. Interactive measures between the operator and screen 18 then are permitted only after activation of the function switch 20.

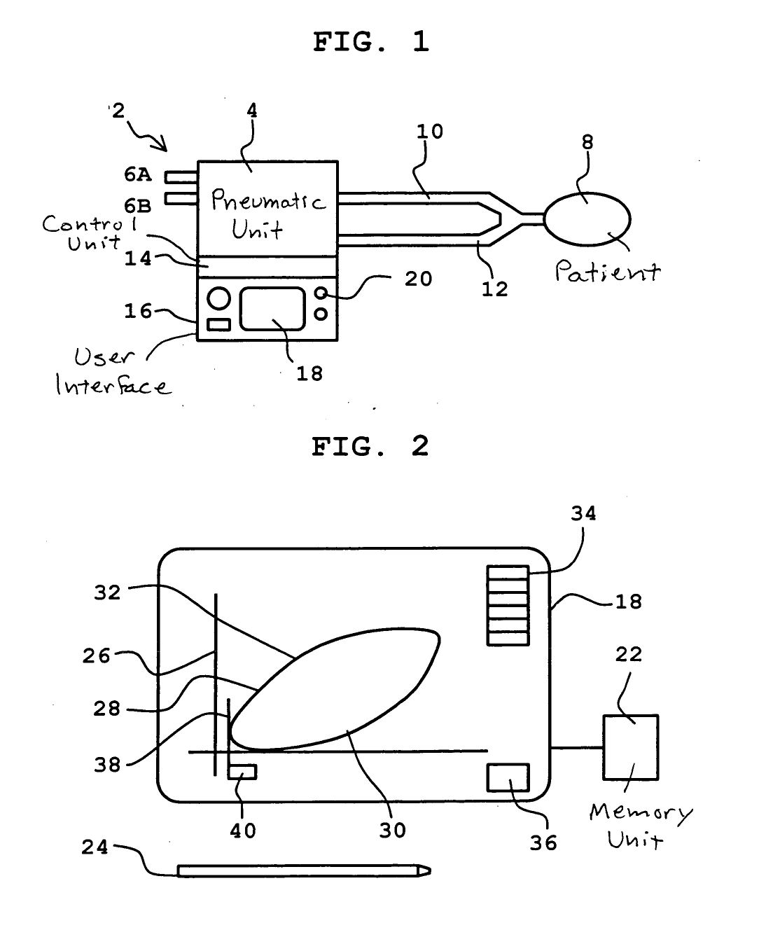

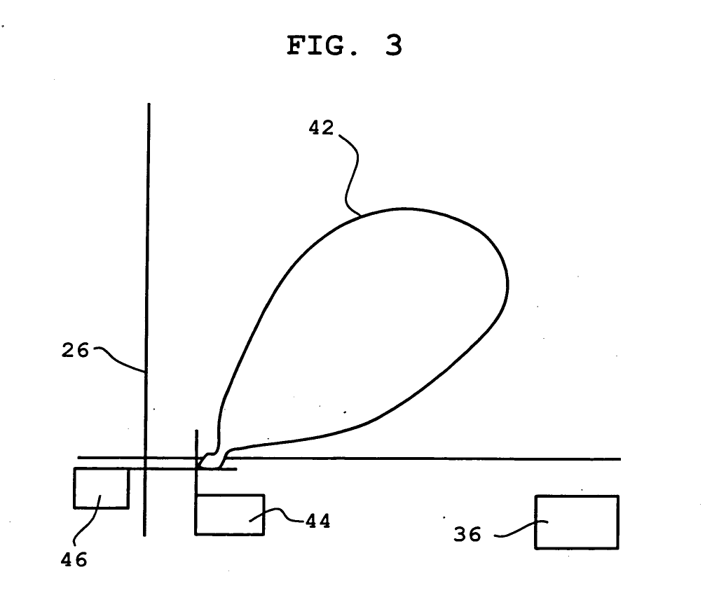

In FIG. 2 the interactive screen 18 is shown more clearly. A memory unit 22 connected to the screen 18 is also shown. The memory unit 22 is also connected to the control unit 14 in FIG. 1 (not shown in the FIG. 2).

The function parameters for the ventilation mode which shall be applied to the patient 8 are stored in the memory unit 22. These include, among other things, ventilation mode, target values for one or more of the parameters: pressure, flow, tidal volume, inspiration time and expiration time, etc. Other parameters may also be found, such as composition of the breathing gas, etc.

The interactive screen 18 of the foregoing exemplary embodiment can be modified by means of a pointer device 24. The pointer device 24 is not essential but does allow a more precise revision of the screen contents than does the use of a finger.

A coordinate system 26 is drawn on the screen 18 as a graphic representation of the actual ventilation mode (corresponding target values in memory unit 22). The x-axis represents pressure and the y-axis represents volume. A curve 28 is displayed in the coordinate system 26. the curve 28 represents a breathing cycle (inspiration 30 and expiration 32).

When programming a ventilator mode, the pointer device 24 (or a finger) may select one mode from a mode list 34 on the screen. Other ways of selecting a mode are also feasible, for instance by pointing at the relevant axis (pressure or volume) to select a mode having the relevant parameter as control parameter—for instance, pointing at the pressure axis may be used to select one of pressure control (PC), pressure support (PS), volume support (VS), pressure regulated volume control (PRVC), continuous positive airway pressure (CPAP) and pointing at the volume axis may be used to select one of volume control (VC), synchronized intermittent mandatory ventilation (SIMV), etc.

The axes can be highlighted in different colors to indicate which mode that presently is set. A selected mode may be verified via an accept button or key 36 on the screen. Other ways of accepting inputs can also be utilized.

Once the mode is selected, parameters have to be set or determined, for instance positive end expiratory pressure (PEEP), peak inspiratory pressure (PIP), maximum allowed overpressure, etc. The parameters that can be set can be highlighted as lines 38 in the graph 26 with default values set in the memory 22 for each ventilator mode. These lines can be displayed with different colors. The user (physician or other permissible user) may then adapt the settings for the present patient by moving the line 38 for each parameter (using the pointer device 24 or a finger). Actual values can easily be read from a numeric information field 40.

Pressing accept button 36 again sets the altered values for the parameters and stores these in the memory unit 22 as target values.

The screen 18 will also display actual measured values during the treatment with the selected ventilation mode. By overwriting the previous curve in a different color or lighting, the physician can easily follow any short term trend or change in the respiratory pattern. By successively dimming two or three previous breaths, the physician will get a better control over minute changes than any time based separate display of pressure and volume.

A trend curve can also be displayed on the screen. The trend curve could be displayed in a different color as a background curve and can comprise the average of a certain number of preceding breaths or over a specific time, e.g. one minute.

Should the measured values move outside target values, the set outer limits can successively be highlighted (possibly simultaneously with the sounding of audible alarms). The physician may then quickly spot which parameter is out of order and quickly take control over the situation.

Some possible functions that can be implemented in the user interface according to the invention are displayed in FIGS. 3 to 5 and described below. The essence of the invention, however, resides in the basic use of the volume-pressure graph as a tool for displaying respiratory curves and inputting target values or other programming.

Thus, in FIG. 3, entering trigger levels for breaths is indicated. Numerals for the graph 26 and accept knob 36 are maintained as they can be identical to the above. A respiration curve 42 is displayed. In order to allow a patient to initiate inspiration phases, trigger values are set. In this case the triggering is based on both pressure and flow. Pressure value for triggering can be set via a first flag 44 and flow value via a second flag 46 (here, “flag” indicates the combination of a line and numeric information field). Instead of flow, a trigger volume could be set.

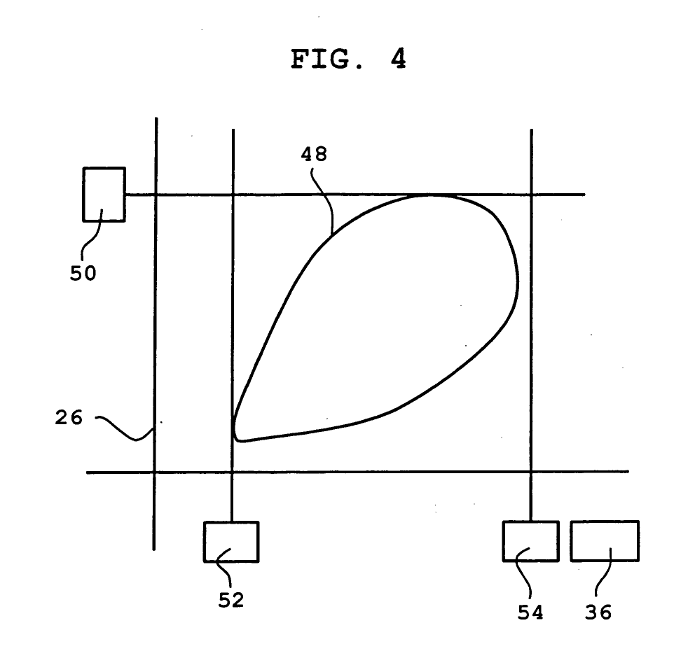

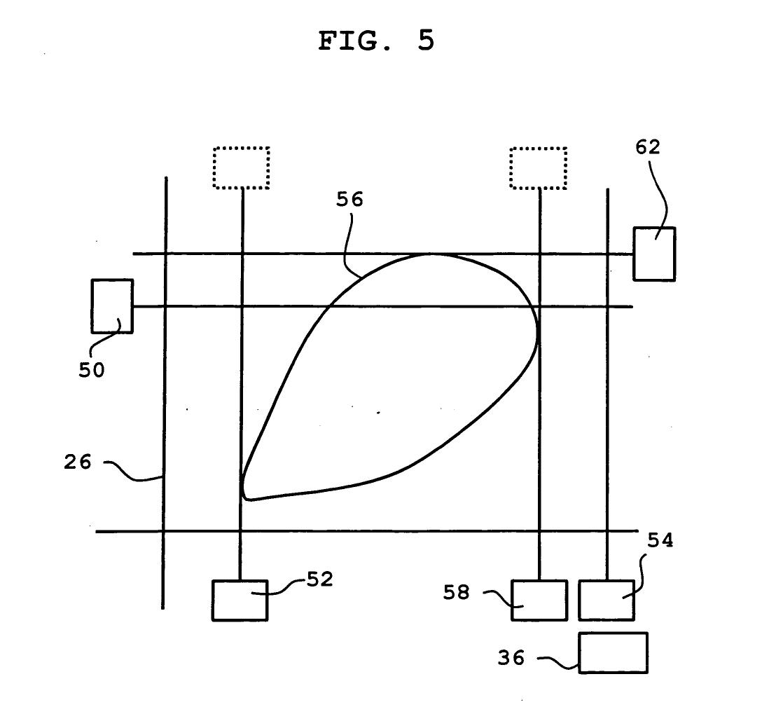

FIG. 4 shows an example of display for a volume control mode. A curve 48 is displayed in the graph 26. the aim in volume control is to achieve a constant tidal volume for each breath (provided with a constant flow of respiratory gas). The main settings here are the tidal volume as represented by tidal volume flag 50 and PEEP as represented by PEEP flag 52. Further, a maximum pressure can also be set, here represented by overpressure flag 54.

As mentioned above, parameters related to the mode itself can be displayed in a different color. In this case, tidal volume flag 50 and PEEP flag 52 would be displayed in different colors than overpressure flag 54 (which relates to safety rather that regulation of the set mode).

Once all parameters are set in accordance with the physician's wishes, accept button 36 can be used to store the set mode (alternately, the user interface can be made such that each set value must be verified before entering the next parameter value).

FIG. 5 shows an example of a display when pressure control is set. Respiration curve 56 represents pressure control. Here, PEEP is set via PEEP flag 52, peak pressure is set via a PIP flag 58 and maximum allowed pressure is set via overpressure flag 54. A minimum value for tidal volume (or minute volume) can be set on the volume axis via minimum tidal volume flag 60. Actual measured values could be displayed in information fields in the area of the graph that displays the curve 56 (whereas all information fields for set parameters are placed on the other side of respective axis). In this case, actual tidal volume (and/or minute volume) is displayed by information flag 62 (similar information can be used for pressures as well, as indicated in the Figure by dashed lines).

Other features not explicitly mentioned above are well known and can be included or can replace certain nonessential features. For instance, numeric display on the volume axis can display current flow value or a small flow curve can be displayed instead.

All breathing apparatus for medical use are included in the context of medical ventilator used in the present application. Accordingly, respirators or ventilators for intensive care, anesthetic apparatus, respirators or ventilators for sub-acute, respirators or ventilators for home care, etc., are all included.

Although modifications and changes may be suggested by those skilled in the art, it is the intention of the inventors to embody within the patent warranted hereon all changes and modifications as reasonably and properly come within the scope of their contribution to the art.

1. A medical ventilator comprising:
a pneumatic unit adapted to interact with a patient for providing respiratory assistance in a mode having a volume and a pressure associated with said respiratory assistance;
a control unit for controlling said pneumatic unit for producing said respiratory assistance;
an input unit connected to said control unit for allowing a user to enter target values for control parameters for said respiratory assistance; and
a display screen connected to said control unit for displaying curves generated by said control unit associated with said respiratory assistance, and said input target values, in a volume-pressure graph.

2. A ventilator as claimed in claim 1 wherein said display screen is an interactive screen and forms said input unit.

3. A ventilator as claimed in claim 2 comprising a pointer device manipulatable by a user for entering said target values via said interactive screen.

4. A ventilator as claimed in claim 1 wherein said control unit divides the volume pressure graph displayed at said display screen with volume values entered along a volume axis and pressure values entered along a pressure axis.

5. A ventilator as claimed in claim 1 wherein said pneumatic unit includes sensors adapted to interact with the patient to obtain measured values associated with said respiratory assistance, and wherein said control unit causes said display screen to display values among said measured values that exceed said target values.

6. A ventilator as claimed in claim 1 wherein said control unit causes a volume axis of said volume-pressure graph to be displayed with a first color and a pressure axis of said volume-pressure graph to be displayed with a second color, different from said first color.
